(19) United States
(12) Patent Application Publication
(10) Pub. No.: US 2012/0029298 A1
(43) Pub. Date: Feb. 2, 2012

(54) Linear classification method for determining acoustic physiological signal quality and device for use therein

(76) Inventors: Yongji Fu, Vancouver, WA (US); Te-Chung Isaac Yang, Aliso Viejo, CA (US); Bryan Severt Hallberg, Vancouver, WA (US)

(21) Appl. No.: 12/804,749

(22) Filed: Jul. 28, 2010

(51) Int. Cl. A61B 5/00

(52) U.S. Cl. 600/300

(57) ABSTRACT

Linear classification is used to determine the quality of acoustic physiological signal samples. A feature dataset is extracted from acoustic physiological signal samples of known quality (i.e., weak, noisy, good) acquired over a sampling period. A linear discriminant analysis is performed on the feature dataset to determine a direction of a linear classifier for the feature dataset. A classification error risk analysis is performed on the feature dataset to determine an offset of the linear classifier. The linear classifier is used to classify into reliability classes acoustic physiological signal samples acquired over an operating period. Information is selected for outputting using the assigned classifications, and is outputted.

### BACKGROUND OF THE INVENTION

The present invention relates to physiological monitoring and, more particularly, to a method for using linear classification to determine the quality (e.g., reliability) of acoustic physiological signal samples and a physiological monitoring device for use in such a method.

Physiological monitoring is in widespread use managing chronic diseases and in elder care. Physiological monitoring is often performed using wearable devices that acquire and analyze acoustic physiological signal samples, such as heart and lung sound samples, as people go about their daily lives. However, these samples are not always reliable. For example, a sample may be too noisy to reliably detect heart or lung sounds if taken when a person speaks, or is in motion, or is in an environment with high background noise. Moreover, a sample may be too weak to reliably detect heart or lung sounds if taken when an acoustic sensor of the monitoring device is not placed at the proper body location or when an air chamber of the acoustic sensor is not fully sealed. When a sample is too noisy or too weak, confidence in physiological data extracted from the sample, such as the patient's heart or respiration rate, may be very low.

Reliance on physiological data extracted from an unreliable physiological signal sample can have serious adverse consequences on patient health. For example, such physiological data can lead a patient or his or her clinician to improperly interpret the patient's physiological state and cause the patient to undergo treatment that is not medically indicated or forego treatment that is medically indicated.

### SUMMARY OF THE INVENTION

The present invention uses linear classification to determine the quality of acoustic physiological signal samples. A feature dataset is extracted from acoustic physiological signal samples of known quality (e.g., weak, noisy, good) acquired over a sampling period. A linear discriminant analysis (LDA) is performed on the feature dataset to determine a direction of a linear classifier for the feature dataset. A classification error risk analysis is performed on the feature dataset to determine an offset of the linear classifier. The linear classifier is used to classify into reliability classes acoustic physiological signal samples acquired over an operating period. Information is selected for outputting using the assigned classifications, and is outputted.

In one aspect of the invention, a method for using linear classification to determine the quality of acoustic physiological signal samples comprises the steps of extracting a feature dataset from first acoustic physiological signal samples of predetermined reliability, determining a linear classifier from the feature dataset, assigning to reliability classes second acoustic physiological signal samples acquired by a physiological monitoring device using the linear classifier, and outputting by the physiological monitoring device information selected using the assigned reliability classes.

In some embodiments, the feature dataset comprises central peak width data for autocorrelation results generated from energy envelopes extracted from the first acoustic physiological signal samples.

In some embodiments, the feature dataset comprises non-central peak amplitude data for autocorrelation results generated from energy envelopes extracted from the first acoustic physiological signal samples.

In some embodiments, the step of determining a linear classifier comprises determining a direction of the linear classifier using a LDA. In some embodiments, the LDA invokes the Fisher method.

In some embodiments, the step of determining a linear classifier comprises determining an offset of the linear classifier using a classification error risk analysis.

In some embodiments, the information comprises a confidence level.

In some embodiments, the information comprises a result reliability indicator.

In some embodiments, the information comprises a recommendation as to how to improve reliability.

In some embodiments, the information is displayed on the physiological monitoring device.

In some embodiments, the extracting and determining steps are performed by the physiological monitoring device.

In some embodiments, the physiological monitoring device is portable.

In another aspect of the invention, a physiological monitoring device comprises a physiological data capture system; a physiological data processing system communicatively coupled with the capture system; and a physiological data output interface communicatively coupled with the processing system, wherein under control of the processing system the device assigns to reliability classes using a linear classifier acoustic physiological signal samples acquired by the device and selects using the assigned reliability classes information respecting the acoustic physiological signal samples, and wherein the information is outputted on the output interface.

In some embodiments, under control of the processing system the device determines the linear classifier from a feature dataset extracted from first acoustic physiological signal samples of predetermined quality.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a physiological monitoring device in some embodiments of the invention.

FIG. 2 shows a linear classification method in some embodiments of the invention.

FIG. 3 shows an exemplary weak acoustic physiological signal sample.

FIG. 4 shows an autocorrelation result for an exemplary weak acoustic physiological signal sample.

FIG. 5 shows an exemplary noisy acoustic physiological signal sample.

FIG. 6 shows an autocorrelation result for an exemplary noisy acoustic physiological signal sample.

FIG. 7 shows an exemplary good acoustic physiological signal sample.

FIG. 8 shows an autocorrelation result for an exemplary good acoustic physiological signal sample.

FIG. 9 shows a feature dataset for acoustic physiological signal samples extracted from autocorrelation results of predetermined reliability.

FIG. 10 shows an alternative representation of the feature dataset of FIG. 9 showing a linear classifier determined for the feature dataset.

FIG. 11 is a display screen displayed to a user of a physiological monitoring device in response to classification of an acoustic physiological signal sample as unreliable in some embodiments of the invention.

FIG. 12 is a display screen displayed to a user of a physiological monitoring device in response to classification of an acoustic physiological signal sample as unreliable in other embodiments of the invention.

### DETAILED DESCRIPTION OF A PREFERRED EMBODIMENT

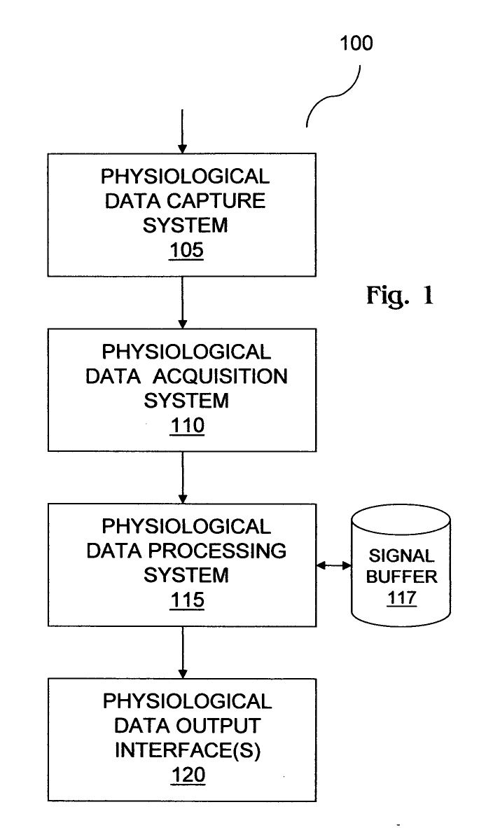

FIG. 1 shows a physiological monitoring device 100 in some embodiments of the invention. Monitoring device 100 includes a physiological data capture system 105, a physiological data acquisition system 110, a physiological data processing system 115 and one or more physiological data output interfaces 120, communicatively coupled in series. Processing system 115 is also communicatively coupled with a signal buffer 117.

Capture system 105 detects body sounds, such as heart and lung sounds, at a detection point, such as a trachea, chest or back of a person being monitored and continually transmits an acoustic physiological signal to acquisition system 110 in the form of an electrical signal generated from detected body sounds. Capture system 105 may include, for example, an acoustic transducer positioned on the body of a human subject.

Acquisition system 110 amplifies, filters, performs analog/digital (AID) conversion and automatic gain control (AGC) on the acoustic physiological signal received from capture system 105, and transmits the signal to processing system 115. Amplification, filtering, A/D conversion and AGC may be performed by serially arranged pre-amplifier, band-pass filter, final amplifier, A/D conversion and AGC stages, for example.

Processing system 115, under control of a processor executing software instructions and/or custom logic, processes the acoustic physiological signal to continually estimate one or more physiological parameters for the subject being monitored. To enable continual estimation of physiological parameters, processing system 115 continually buffers in signal buffer 117 and evaluates samples of the acoustic physiological signal, wherein each sample has a sampling window length, such as fifteen seconds. Processing system 115 under control of the processor transmits to one or more output interfaces 120 recent estimates of the monitored physiological parameters and other information for display or further processing.

Output interfaces 120 includes a user interface having a display screen for displaying recent estimates of monitored physiological parameters and other information in accordance with format and content information received from processing system 115. Output interfaces 120 may also include a data management interface to an internal or external data management system that stores the estimates and information and/or a network interface that transmits the estimates and information to a remote monitoring device, such as a monitoring device at a clinician facility.

In some embodiments, monitoring device 100 is a portable ambulatory monitoring device that monitors a person's physiological well-being in real-time as the person performs daily activities. In other embodiments, capture system 105, acquisition system 110, processing system 115 and output interfaces 120 may be part of separate devices that are remotely coupled via wired or wireless links.

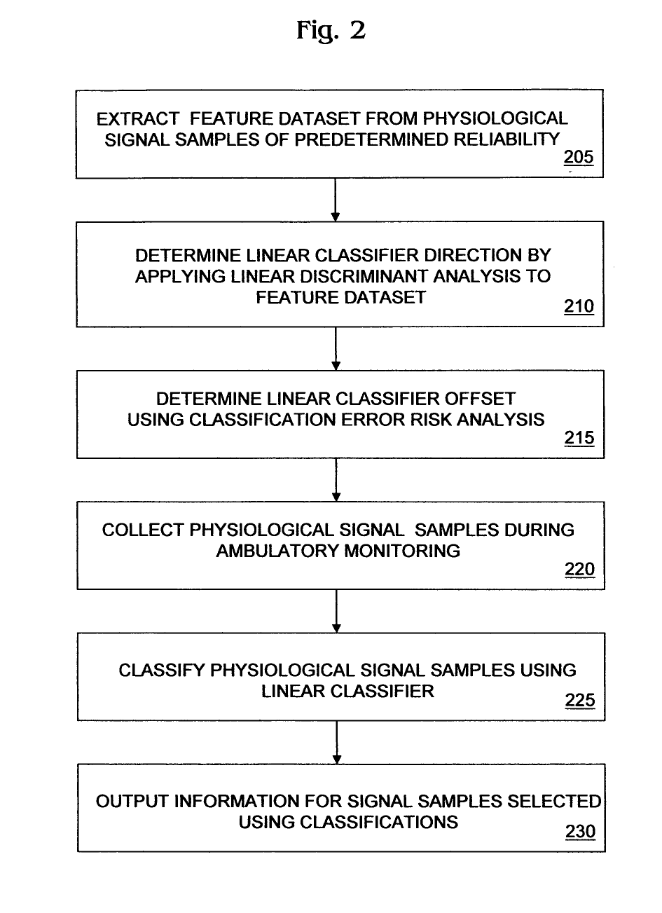

FIG. 2 shows a linear classification method in some embodiments of the invention. Steps 205-215 of the method relate to determining a linear classifier, whereas Steps 220-230 of the method relate to using the linear classifier during operation of monitoring device 100 to assess the reliability of physiological signal samples in real-time. In some embodiments, Steps 205-215 are performed remotely from monitoring device 100 and the linear classifier is preconfigured on monitoring device 100 without regard to the user's individual physiology or operating environment. In other embodiments, Steps 205-215 are performed on monitoring device 100 and the linear classifier is tailored to the user's individual physiology and/or operating environment. In the discussion that follows, it is assumed that Steps 205-215 are performed on monitoring device 100 under control of a processor running on processing system 115.

Consider, for example, a situation where it is desired to estimate heart rate from an acoustic physiological signal. In that event, the linear classification method proceeds as follows: At Step 205, a feature dataset is extracted from acoustic physiological signal samples of predetermined reliability. For this, monitoring device 100 is exposed to environments wherein capture system 105 detects weak, noisy and good samples and processing system 115 builds a feature dataset from autocorrelation results for the weak, noisy and good samples. Three components are recorded for each sample in the feature dataset: (1) reliability, (2) amplitude of the highest non-central autocorrelation peak centered between 0.33 seconds and 1.5 seconds (which corresponds to the typical human heartbeat period of between 0.33 and 1.5 seconds) and (3) half-width of the autocorrelation peak centered at zero time delay. The reliability of each sample is presumed from the environment in which the sample is acquired. For example, a sample is presumed to be unreliable if capture system 105 is placed away from the body of the person being monitored and/or large background noise is present when the sample is detected, whereas a sample is presumed to be reliable if capture system 105 is correctly placed on the body of the person being monitored and background noise is absent when the sample is detected. The non-central peak amplitude and central peak width of the autocorrelation result are chosen as features for the feature dataset since reliable signals differ in a statistically significant manner from unreliable signals with regard to these two features, as will now be discussed in connection with FIGS. 3-8.

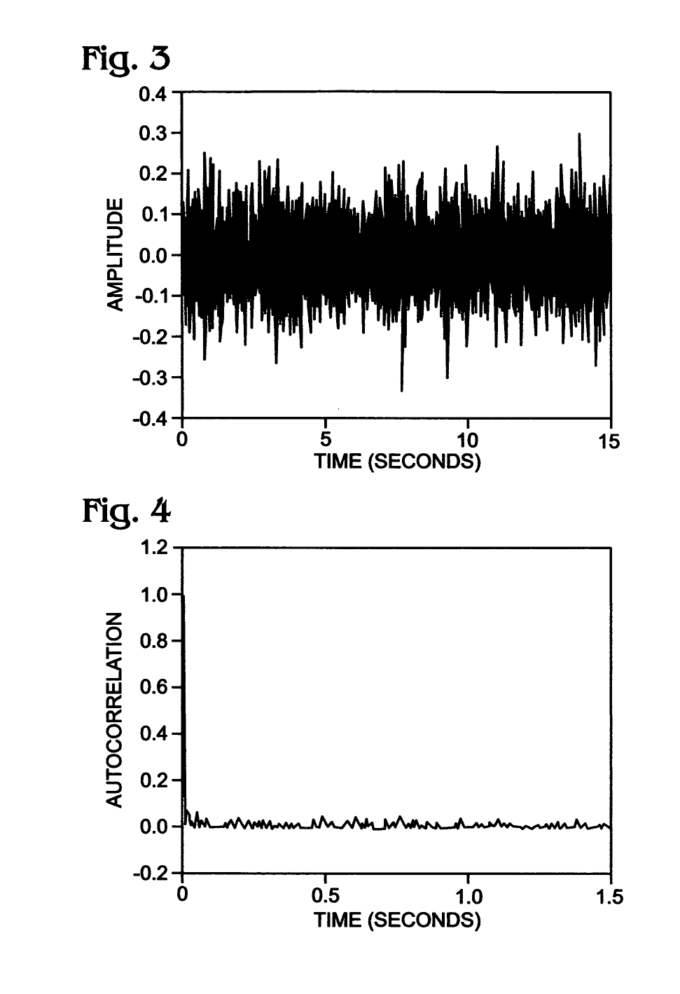

FIG. 3 shows an exemplary weak tracheal acoustic physiological signal sample. Such a sample may be acquired by, for example, placing an acoustic transducer of capture system 105 away from the body of the person being monitored. The illustrated sample was acquired over fifteen seconds. The X-axis is time in seconds and the Y-axis is signal amplitude in aptitude units. The sample includes several body sounds and noise from different sources. The body sounds in the sample are weak throughout the sampling window, making them difficult to isolate. At processing system 115, a band-pass filter is applied to the sample to better isolate body sounds of interest. As heart sounds are typically found within the 20 to 120 Hz frequency range, a band-pass filter having a cutoff frequency of 20 Hz at the low end and 120 Hz at the high end is applied to the sample to isolate heartbeat. An energy envelope is then extracted from the sample to further remove noise and improve signal quality. The energy envelope can be extracted using, for example, a standard deviation method. Finally, an autocorrelation function is applied to the energy envelope to identify any fundamental periodicity in the sample. An autocorrelation result for the sample is shown in FIG. 4. The autocorrelation result is characterized by the absence of any significant central peak (i.e., peak centered at zero time delay) and the absence of any significant non-central peak (i.e., peak centered between 0.33 and 1.5 second time delay), reflecting a sample wherein heartbeat is largely nonexistent due to weak detection. This weak detection prevents heart rate data from being reliably extracted from the sample, such that the sample is unreliable.

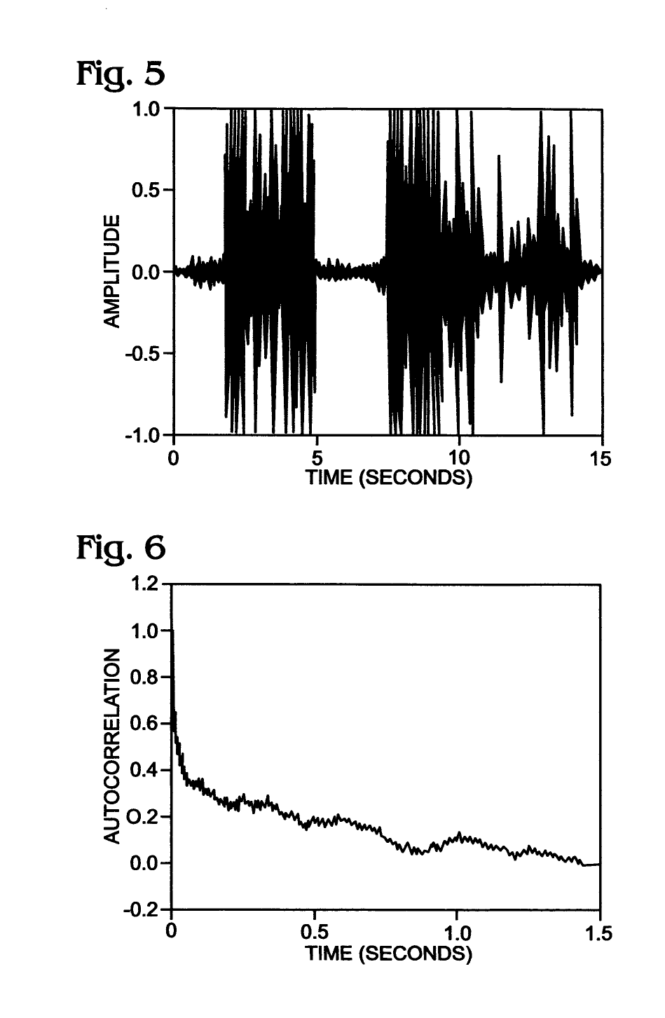

FIG. 5 shows an exemplary noisy tracheal acoustic physiological signal sample. Such a sample may be acquired by, for example, introducing large background noise into the environment of the person being monitored. The illustrated sample was again acquired over fifteen seconds and the X-axis is again time in seconds and the Y-axis is signal amplitude in aptitude units. The sample again includes several body sounds and noise from different sources. However, the sample is disrupted by strong noise in portions of the sampling window, making it difficult to isolate body sounds, such as heartbeat, in the sample. A band-pass filter having a cutoff frequency of 20 Hz at the low end and 120 Hz at the high end is applied to the signal sample to isolate heartbeat. An energy envelope is extracted from the sample to further remove noise and improve signal quality. Finally, an autocorrelation function is applied to the energy envelope to identify any fundamental periodicity in the sample. As shown in FIG. 6, the autocorrelation result is characterized by a central peak having a large width, reflecting a sample whose periodic energy (i.e., heartbeat) is largely subsumed in higher energy noise. This noise prevents heart rate data from being reliably extracted from the sample, such that the sample is unreliable.

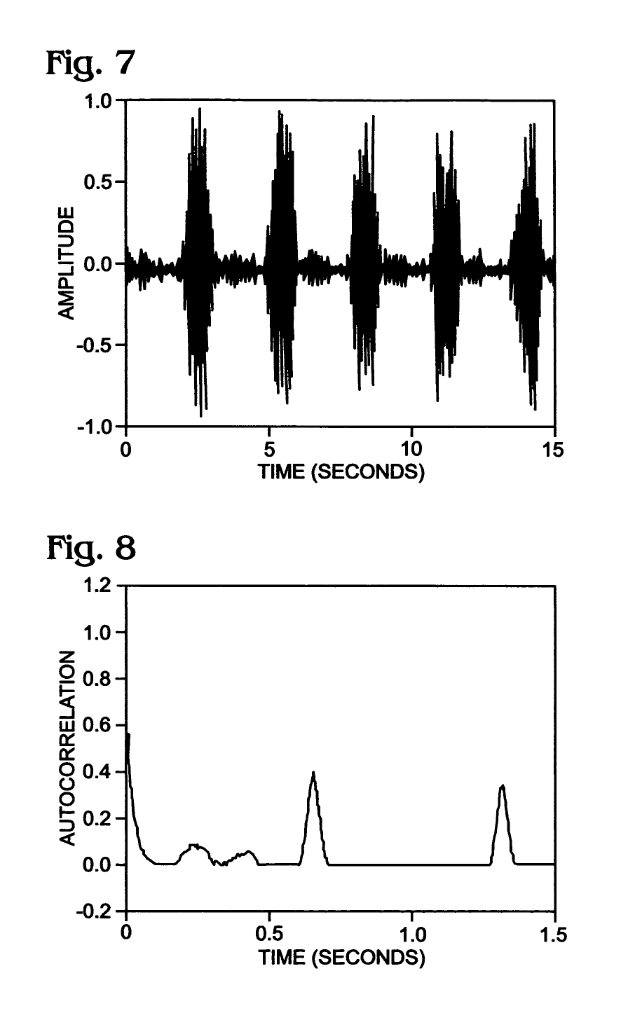

FIG. 7 shows an exemplary good tracheal acoustic physiological signal sample. Such a sample may be acquired by proper placement of an acoustic transducer on the person being monitored and a quiet environment. The illustrated sample was again acquired over fifteen seconds and the X-axis is again time in seconds and the Y-axis is signal amplitude in aptitude units. The sample again includes several body sounds and noise from different sources. A band-pass filter having a cutoff frequency of 20 Hz at the low end and 120 Hz at the high end is applied to the sample to isolate heartbeat.

An energy envelope is extracted from the sample to further remove noise and improve signal quality. Finally, an autocorrelation function is applied to the energy envelope to identify fundamental periodicity in the sample. As shown in FIG. 8, the autocorrelation result is characterized by significant signal peaks, including a central peak centered at zero time delay and a non-central peak centered between 0.33 and 1.5 seconds from which heart rate data can be reliably extracted. The non-central peak centered at about 0.7 seconds corresponds to a heart rate of roughly 85 beats per minute (60/0.7=85.7).

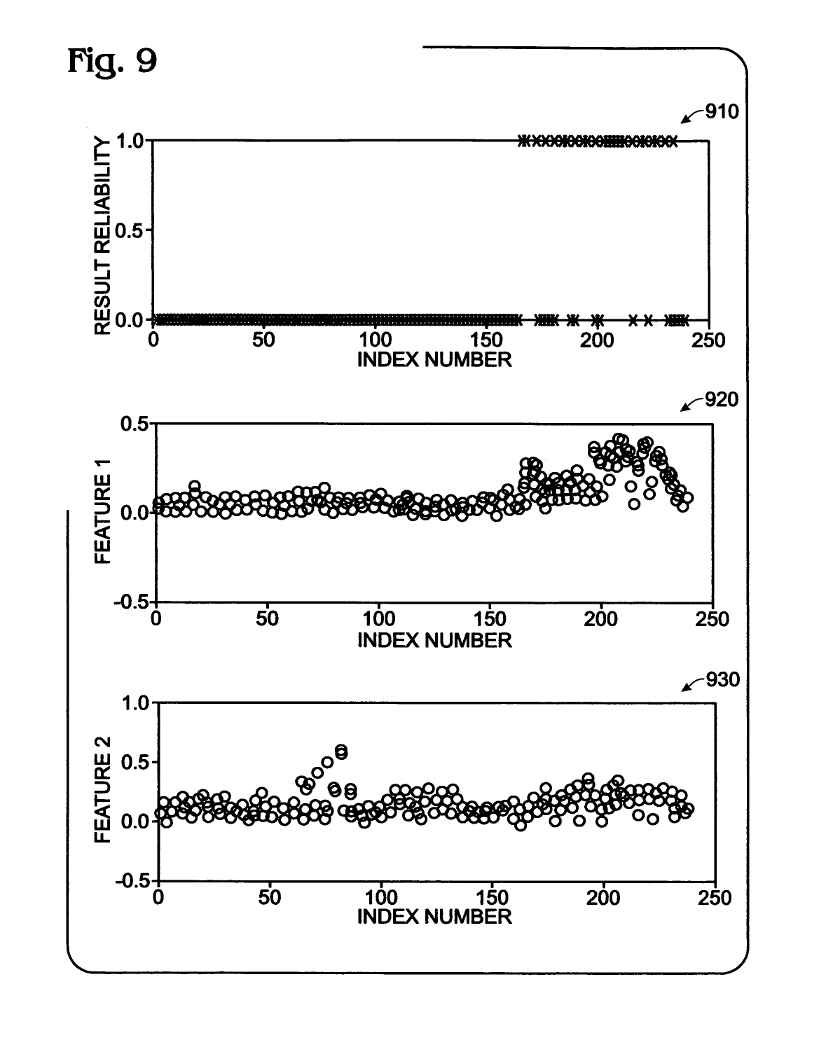

FIG. 9 shows an exemplary feature dataset extracted from samples of varying predetermined reliability over a sampling period. The feature dataset includes hundreds of samples of known reliability, including (unreliable) weak signal samples, (unreliable) noisy signal samples and (reliable) good signal samples. Plot 910 plots the presumed reliability of each sample taken over the sampling period. For example, samples 1-150 are presumed unreliable (and assigned a reliability value of “0”) due to placement of the acoustic transducer away from the body of the person being monitored and/or introduction of large background noise when those samples were taken, whereas certain samples between 151 and 250 are presumed reliable (and assigned a reliability value of “1”) due to correct placement of the acoustic transducer on the body of the person being monitored and suspension of background noise when those samples were acquired. Plot 920 shows the non-central peak amplitude (Feature 1) of each sample taken over the sampling period. As can be seen, the non-central peak amplitude is typically at or near zero for unreliable signal samples and significantly above zero for reliable signal samples. Plot 930 shows the central peak half-width (Feature 2) of each sample taken over the sampling period. As can be seen, the central peak half-width is typically either near zero or substantially above zero for unreliable signal samples and more modestly above zero for reliable signal samples. A linear classifier is determined for the feature dataset and used to classify further acoustic physiological signal samples acquired during physiological monitoring of a person being monitored, as will now be explained in even greater detail.

At Step 210, a line direction of a linear classifier for the feature dataset is determined using a LDA. The Fisher method may be used, by way of example, in which the selected line direction is perpendicular to ν, wherein ν is computed according to the formula

ν=S_{w}⁻¹(μ₁−μ₂)

wherein μ₁ is the mean for the reliable class, μ₂ is the mean for the unreliable class and S_{w} is the within class scatter.

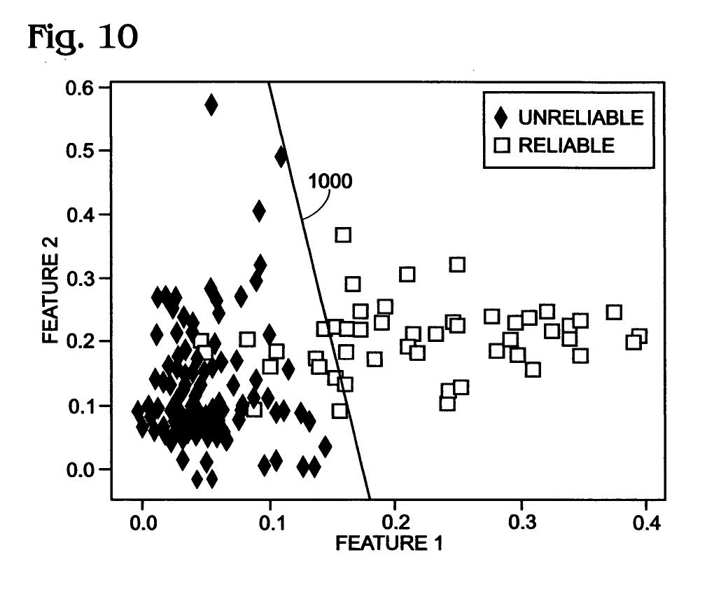

At Step 215, a positional offset of the linear classifier is determined using a classification error risk analysis. Application of a linear classifier over a sustained period will result in inevitable errors in classification (i.e., false positives and false negatives). In some embodiments, the offset of the linear classifier is selected to equalize the number of false positives and false negatives. In other embodiments, consideration is given to the fact the adverse consequences arising from false positives and false negatives may differ in severity. For example, inducing action based on an unreliable sample erroneously classified as reliable may be more adverse to health outcomes than inducing non-action on a reliable sample erroneously classified as unreliable. Accordingly, the offset of the linear classifier in some embodiments may be selected such that the share of erroneous classifications of an unreliable signal sample as reliable is smaller than the share of erroneous classifications of a reliable signal as unreliable. FIG. 10 is an alternative representation of the feature dataset of FIG. 9 showing a linear classifier 1000 selected for that feature dataset. An offset has been selected such that all unreliable signal samples are correctly classified, whereas a number of reliable signal samples are classified as unreliable. Linear classifier 1000 is stored on monitoring device 100 by processing system 115 under control of a processor and is referenced during subsequent ambulatory monitoring over a sustained operating period as set forth in Steps 220-230, which are performed by processing system 115 under control of a processor.

At Step 220, acoustic physiological signal samples are acquired by device 100 during an operating period. For each sample, a window of the acoustic physiological signal of a current sample window length is stored in signal buffer 117. In this raw signal, lung sounds are intermingled with heart sounds and noise and are not easily distinguished. A band-pass filter is applied to the sample to better isolate heart sounds by reducing lung sounds and noise. An energy envelope is extracted from the sample to further improve signal-to-noise ratio. In some embodiments, a standard deviation method is used to extract the energy envelope. An autocorrelation function is applied to the energy envelope to identify fundamental periodicity in the sample. The non-central peak amplitude and central peak width (i.e., half-width) are recorded for each sample.

At Step 225, the samples are classified using linear classifier 1000. Returning to FIG. 10, if the non-central peak amplitude and the central peak width for a sample form a coordinate that falls on the right of linear classifier 1000, the sample is classified as reliable. On the other hand, if the non-central peak amplitude and the central peak width for the sample form a coordinate that falls on the left of linear classifier 1000, the sample is classified as unreliable.

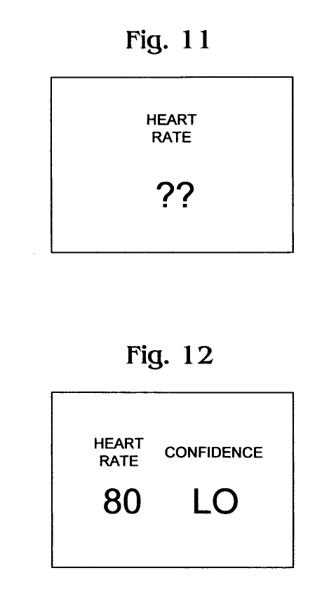

At Step 230, classification dependent information for the samples is selected and outputted by processing system 115 on one or more of output interfaces 120. In some embodiments, if a sample has been classified as reliable, a heart rate estimate is extracted from the sample and transmitted to a user interface whereon the heart rate estimate is displayed to the person being monitored. On the other hand, if a sample has been classified as unreliable, a heart rate estimate may or may not be extracted from the sample or displayed. Moreover, information indicative of reliability may be displayed. For example, in FIG. 11 a display screen displayed on a user interface in response to classification of a sample as unreliable is shown in some embodiments of the invention. The display screen displays question marks in lieu of a heart rate estimate extracted from the sample to prevent reliance by the person being monitored on a potentially unreliable estimate. In FIG. 12, a display screen displayed on a user interface in response to classification of a sample as unreliable is shown in other embodiments of the invention. The display screen displays the heart rate estimate and also displays a confidence level indicating that confidence in the estimate is low. Other classification dependent information may be outputted on a user interface, such as a recommendation as to corrective action to improve signal quality, such as “relocate transducer” or “move to quieter environment.” Furthermore, in addition to or in lieu of display of information on a user interface, information may be transmitted to one or more of a local analysis module whereon a heart rate estimate is subjected to higher level clinical processing, a data management element whereon the estimate is logged, and/or transmitted to a network interface for further transmission to a remote analysis module or remote clinician display.

It will be appreciated by those of ordinary skill in the art that the invention can be embodied in other specific forms without departing from the spirit or essential character hereof. In one variant, a feature dataset may include three or more features and multiple discriminant analysis (MDA) may be used to determine a classifier. In another variant, classification may result in action in addition to or in lieu of outputting of information, such as adding an extra noise elimination step in signal processing.

The present description is therefore considered in all respects to be illustrative and not restrictive. The scope of the invention is indicated by the appended claims, and all changes that come with in the meaning and range of equivalents thereof are intended to be embraced therein.

What is claimed is:

1. A method for using linear classification to determine the quality of acoustic physiological signal samples, comprising the steps of:
extracting a feature dataset from first acoustic physiological signal samples of predetermined reliability;
determining a linear classifier from the feature dataset;
assigning to reliability classes second acoustic physiological signal samples acquired by a physiological monitoring device using the linear classifier; and
outputting by the physiological monitoring device information selected using the assigned reliability classes.

2. The method of claim 1, wherein the feature dataset comprises central peak width data for autocorrelation results generated from energy envelopes extracted from the first acoustic physiological signal samples.

3. The method of claim 1, wherein the feature dataset comprises non-central peak amplitude data for autocorrelation results generated from energy envelopes extracted from the first acoustic physiological signal samples.

4. The method of claim 1, wherein the step of determining a linear classifier comprises determining a direction of the linear classifier using a linear discriminant analysis (LDA).

5. The method of claim 4, wherein the LDA invokes the Fisher method.

6. The method of claim 1, wherein the step of determining a linear classifier comprises determining an offset of the linear classifier using a classification error risk analysis.

7. The method of claim 1, wherein the information comprises a confidence level.

8. The method of claim 1, wherein the information comprises a reliability indicator.

9. The method of claim 1, wherein the information comprises a recommendation as to how to improve reliability.

10. The method of claim 1, wherein the information is displayed on the physiological monitoring device.

11. The method of claim 1, wherein the extracting and determining steps are performed by the physiological monitoring device.

12. The method of claim 1, wherein the physiological monitoring device is portable.

13. A physiological monitoring device, comprising:
a physiological data capture system;
a physiological data processing system communicatively coupled with the capture system; and
a physiological data output interface communicatively coupled with the processing system, wherein under control of the processing system the device assigns to reliability classes using a linear classifier acoustic physiological signal samples acquired by the device and selects using the assigned reliability classes information respecting the acoustic physiological signal samples, and wherein the information is outputted on the output interface.

14. The device of claim 13, wherein under control of the processing system the device determines the linear classifier from a feature dataset extracted from acoustic physiological signal samples of predetermined reliability.

15. The device of claim 14, wherein the feature dataset comprises central peak width data for autocorrelation results generated from energy envelopes extracted from the first acoustic physiological signal samples.

16. The device of claim 14, wherein the feature dataset comprises non-central peak amplitude data for autocorrelation results generated from energy envelopes extracted from the first acoustic physiological signal samples.

17. The device of claim 13, wherein a direction of the linear classifier is determined using a LDA.

18. The device of claim 13, wherein an offset of the linear classifier is determined using a classification error risk analysis.

19. The device of claim 13, wherein the information is displayed on the output interface.

20. The device of claim 13, wherein the device is portable.
